(19) United States
(12) Patent Application Publication
(10) Pub. No.: US 2008/0213244 A1
(43) Pub. Date: Sep. 4, 2008

(54) Glutamate Receptor Antagonists as Neuroprotectives

(76) Inventor: Wolfgang Sohngen, Aachen (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/555,583

(22) PCT Filed: May 5, 2004
(86) PCT No.: PCT/EP2004/004776

§ 371 Date: Oct. 16, 2006

(30) Foreign Application Priority Data
May 5, 2003 (DE) 103 37 098.6
May 6, 2003 (DE) 103 20 336.2
Nov. 6, 2003 (DE) 103 52 333.2

(51) Int. Cl. A61K 38/48; A61K 38/17; A61P 25/16; A61P 25/28

(52) U.S. Cl. 424/94.64; 424/94.63; 514/25

(57) ABSTRACT

The invention relates to the use of inhibiting agents of t-PA-mediated activation of the glutamate receptor, for example of the NMDA type, as neuroprotectives.

The invention relates to neuroprotectives for the therapy and prophylaxis of neurological damage, in particular of damage indirectly or directly attributable to overactivation of the glutamate receptor of the methyl-D-asparate type (NMDA-glutamate receptor hereinafter) and claims the priority of the German patent applications 103 37 098.6, 103 20 336.2 and 103 52 333.2, which are incorporated herein by reference.

The term “neuroprotectives” means in the context of the present invention therapeutic agents or active ingredients which contribute to protecting nerve cells from cell damage and/or to counteracting neurodegenerative processes and impairments of neuronal efficiency.

In the development of neuroprotectives, substantial efforts are directed in particular at the identification and/or further development of glutamate receptor antagonists which protect nerve cells against an increased activity of excitatory neurotransmitter receptors (excitotoxicity) or moderate the overactivation. The most important group of receptor antagonists are the NMDA antagonists which inhibit the NMDA type of the glutamate receptor. A distinction is made in this connection between the following agonists: competitive NMDA antagonists such as 2-amino-5-phosphonopentanoate ((D)-AP5) and (±)-4-(4-phenyl-benzoyl)piperazine-2,3-dicarboxylic acid (PBPD), which act on the glutamate binding site, NMDA receptor channel blockers such as MK-801, memantine and ketamine, which block the ion channel of the receptor, the NMDA antagonists which act on the glycine binding site, such as GV96771A, which inhibit the binding of the coagonist glycine, and the polyamine site antagonists such as ifenprodil, which inhibit the binding receptor-stimulating polyamines.

Three classes of glutamate receptors (AMPA, kainate and NMDA receptors), each of which act as glutamate-dependent ion channels, conduct the post-synaptic signal of the most important excitatory neurotransmitter glutamate. NMDA-glutamate receptors are disseminated throughout the brain in the form of different receptor subtypes and are essential as crucial excitatory neurotransmitter receptors for the function of the central nervous system (CNS).

There has in recent years been detailed investigation of the molecular biology of the NMDA-glutamate receptor. It emerged from this that these receptors consist of an NR1 subunit in combination with one or more NR2 subunits and—less commonly—an NR3 subunit (Das, S. et al.: Increased NMDA current and spine density in mice lacking the NMDA receptor subunit NR3A. Nature 393 (1998), 377-381; Chatterton, J. E. et al.: Excitatory glycine receptors containing the NR3 family of NMDA receptor subunits. Nature 415 (2002), 793-798).

The NR2 subunit, of which the four forms A-D exist, in particular, but also alternative splice variants of the NR1 subunit determine the pharmacology of the NMDA-glutamate receptor. NR1 and NR2A are ubiquitously expressed in the CNS, whereas NR2B, NR2C and NR2D occur less commonly or only in certain regions of the brain. This difference in the composition of the receptor subtypes makes therapeutic approaches possible with selectively acting antagonists.

Responsible for the complex activation of the NMDA-glutamate receptor are glutamate which is released presynaptically as excitatory neurotransmitter, and glycine as modulating neurotransmitter and coagonist. Activation of the receptor leads to an influx of calcium ions and is—especially when stimulation is prolonged—associated with an activation of complex cellular signals. These lead inter alia to a phosphorylation of the CRE binding protein (CREB) and to a multiple gene activation and synaptic plasticity (neuroplasticity), which represents an essential basis for memory and learning processes.

A review of the general pharmacology of the NMDA receptor and of NMDA antagonists is given in the publication by Kemp, J. A. and McKernan, R. M.: NMDA Receptor Pathways as Drug Targets, Nature Neuroscience, November 2002, Vol. 5 Suppl., 1039-1042, which is incorporated herein by reference.

An overactivation of the receptor and the increased Ca²⁺ influx associated therewith leads by contrast to an extensive interference with the Ca²⁺-dependent cellular metabolism—including energy metabolism. Activation of Ca²⁺-dependent catabolic enzymes is thought in particular to be responsible for the cell death which occurs subsequently (Lee, Jin-Moo et al., “The changing landscape of ischemic brain injury mechanisms”; Dennis W. Choi “Glutamat neurotoxicity and diseases of the nervous system”). This process is known as excitotoxicity.

Since excitotoxicity is based on overactivation of the glutamate receptor, in general antagonists of the glutamate receptor have a therapeutic and/or prophylactic potential for all CNS disorders or neuronal conditions in which neurons are threatened or damaged by this process. These “conditions” likewise include the cerebral ischemia occurring during a stroke, as well as neurodegenerative disorders such as Parkinsonism and Huntington's chorea, where the essential cause of the disease is not excess glutamate but an increased sensitivity to excitotoxic damage. Finally, disorders such as epilepsy and neuropathic pain which are based on an overactivity of excitatory signal pathways are also possible areas of use of glutamate receptor antagonists.

Other uses extend for example to the treatment of painful conditions, especially chronic pain, the treatment of addictive disorders, the treatment of psychiatric disorders and the stimulation of learning and memory.

Antagonists are capable of competitive or non-competitive inhibition of the receptor. Whereas the competitive antagonists counteract receptor activation for example by the natural agonists glutamate and glycine, the non-competitive antagonists inhibit the receptor irrespective of the presence or concentration of the agonists—for example by blocking the ion channel. A competitive inhibition (antagonism) of the glutamate receptor of the NMDA type is possible for example with 2-amino-5-phosphonovalerate (APV) or 2-amino-5-phosphonoheptanoates (APH). Non-competitive inhibition can by contrast be achieved by substances which bind to the phencyclidine side of the channels, such as phencyclidine, MK-801, dextrorphan or ketamine.

However, clinical studies to date on the use of NMDA antagonists as neuroprotectives, in particular in cases of stroke and traumatic brain injury, have not yet had the desired result (Kemp, J. A.; Kew, J. N. C.; Gill, R.: Handbook of Experimental Pharmakology Vol. 141 (eds. Jonas, P. & Monyer, H.) 495-527 (Springer, Berlin, 1999); Lees, K. R. et al.: Glycine antagonist (gavestinel) in neuroprotection (GAIN International) in patients with acute STROKE: a randomised controlled trial. GAIN International Investigators. Lancet 355 (2000), 1949-1954; Sacco, R. L. et al. Glycine antagonist in neuroprotection for patients with acute stroke: GAIN Americas: a randomized controlled trial. JAMA 285 (2001), 1719-1728).

The lack of success to date in attempts at neuroprotection in cases of stroke can be attributed inter alia presumably to the fact that the known neuroprotectives must necessarily be combined with thrombolytics in order to penetrate to the site of the tissue damage and display their effect there. It is moreover not normally possible to administer sufficiently high doses of the known NMDA-glutamate receptor antagonist in order to achieve the desired effect, because of their considerable side effects. This is because they cause, in high dosages, inter alia hallucinations and a marked increase in the blood pressure. The latter is of course particularly critical especially for stroke patients.

In addition, the known glutamate receptor antagonists often have, in the dosage necessary for neuroprotection, an unacceptably strong anesthetic or narcotic effect. Thus, many NMDA antagonists such as phencyclidine and ketamine were originally developed for the purpose of anesthesia.

Attempts have therefore been made to avoid these side effects with receptor-selective NMDA antagonists. It emerged from these that the NR2B-selective antagonist ifenprodil has advantageous effects with distinctly reduced side effects in the animal model of stroke (Gotti, B. et al. Ifenprodil and SL 82.0715 as cerebral anti-ischemic agents. Evidence for efficacy in models of focal cerebral ischemia. J. Pharmacoil. Exp. Ther. 247 (1988), 1211-1221). A number of further antagonists of the NR2B subunit, e.g. CP-101606, Ro 25-6981 and Ro 63-1908, has been described since then (Kemp, J. A.; Kew, J. N. C.; Gill, R.: Handbook of Experimental Pharmakology Vol 141 (eds. Jonas, P. & Monyer, H.) 495-527 (Springer, Berlin, 1999); Gill R. et al.: Pharmacological characterization of RO63-1908 (1-[2-(4-hydroxy-phenoxy)-ethyl]-4-(4-methyl-benzyl)-piperidin-4-01), a novel sinotype-selective N-methyl-D-asparatate antagonist. J. Pharmacol. Exp. Ther. 302 (2002), 940-948). These also certainly showed a neuroprotective effect with few side effects in the animal model. However, these positive results could not be confirmed in clinical studies on traumatic brain injury. Rather, CP-101606 repeatedly failed (Press release from Pfizer, October 2001). In addition, a particular problem associated with the search for a suitable neuroprotective for reducing cell damage in stroke is that the neuroprotectives must be combined with a fibrinolytic/thrombolytic in order to overcome the barrier of the thrombus and penetrate into the region of the damaged tissue (see above). The thrombolytic normally administered is t-PA which is the only thrombolytic currently authorized for the treatment of stroke.

However, it is known from the investigations by Nicole et al. that t-PA plays an important part in excitotoxicity (Nicole O; Docagne F Ali C; Margaill I; Carmeliet P; MacKenzie E T; Vivien D and Buisson A. 2001; The proteolytic activity of tissue-plasminogen activator enhances NMDA receptor-mediated signaling; in: Nat Med 7, 59-64). According to this, depolarized cortical neurons secrete t-PA which interacts with the NR1 subunit of the glutamate receptor of the NMDA type and cleaves it. This is associated with an activation of the receptor activity. Administration of t-PA thus contributes to the cell-damaging excitotoxicity. The consequence is a neurotoxic effect of t-PA. Development of a neuroprotective for the prophylaxis and treatment of cell damage associated with stroke is thus confronted by the particular challenge of not only moderating the cell damage caused by the stroke, but also where possible of taking account of the damage caused or enhanced by administration of the therapeutic agent to reopen the vessels.

It is thus the object of the present invention to provide an alternative possibility for the treatment and prophylaxis of neurological damage.

This object is achieved by the use as neuroprotectives of substances which inhibit t-PA activity. The term “inhibition” encompasses in this connection all effects leading to diminution or reduction in t-PA activity. This may involve for example competitive or non-competitive inhibition, accelerated degradation of the t-PA or else a reduction (suppression) in t-PA expression. The term inhibitor correspondingly refers to all substances which cause a reduction in t-PA activity in the cell.

t-PA activity is defined in particular as activation of the glutamate receptor, preferably of the glutamate receptor of the NMDA type. The reduction in the activation of the glutamate receptor by the neuroprotective is preferably measured by determining the Ca⁺⁺ influx into the cells of the affected tissue on administration of the neuroprotective. An assay for visualizing Ca⁺⁺ is known to the skilled worker and is indicated in example 3 in section II.

t-PA inhibitors are extensively known. Thus, for example, it is known that the activity of t-PA is regulated by the plasminogen activator inhibitor (PAI). Likewise, t-PA can be inhibited by the proteases neuroserpin or protease nexin I (PN-1). These inhibitors are in each case serine protease inhibitors which inhibit t-PA in the physiological context. However, they are employed according to the invention as neuroprotectives.

It is also possible according to the invention to reduce the t-PA activity indirectly via an increase in the transcription rate of the physiological inhibitors. Thus, it is possible to administer for example TGF-β (transforming growth factor β) which is known to stimulate transcription of PAI-1.

In a particularly preferred embodiment of the invention, the plasminogen activating factor DSPA (desmoteplase) is employed as neuroprotective, in particular for the treatment of (pathological) conditions derived from excitotoxicity. DSPA with its isoforms is described in detail inter alia in the patents U.S. Pat. No. 5,830,849 and U.S. Pat. No. 6,008,019. Recombinant preparation of DSPA is disclosed in U.S. Pat. No. 5,731,186. These publications are incorporated herein by reference for purposes of disclosing the structure, the function and the provision of DSPA.

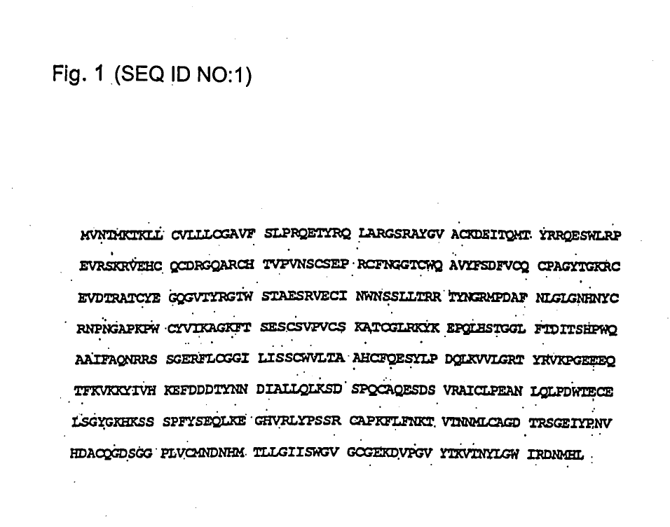

The primary structure of a desmoteplase isoform which is particularly preferably used is depicted in FIG. 1 (DSPA alpha1). However, it is also possible according to the invention to employ other DSPA isoforms having the same function. These are likewise disclosed in the abovementioned U.S. patents. These plasminogen activating factors are referred to hereinafter uniformly as DSPA or desmoteplase without a restriction to DSPA alpha1 being associated therewith.

It is possible according to the invention to employ both native purified DSPA and recombinant DSPA. It is likewise possible to employ derivatives or fragments of DSPA as long as they show the neuroprotective effects of DSPA. The term “DSPA” is therefore understood herein to be the generic term for native or recombinant DSPA and the derivatives, analogs or fragments thereof having substantially the same function.

The terms “derivatives, analogs or fragments” of DSPA subsume in particular all proteins or peptides which functionally display the characteristic properties of native DSPA, especially the increased fibrin specificity in relation to native t-PA. The increased fibrin specificity of DSPA in relation to t-PA is disclosed in WO 03/037363. The DSPA derivatives and analogs preferably have a homology of at least 70%, preferably of at least 80-90%, with the amino acid sequence of DSPA shown in FIG. 1.

The surprising neuroprotective effect according to the invention of DSPA was identified in animal experiments and in in vitro experiments in which it was possible to show that DSPA not only had no neurotoxic but also has a neuroprotective effect since it counteracts the neurotoxic effect of t-PA. It was realized from this that DSPA acts as antagonist of t-PA. It was further possible to show that high concentrations of DSPA lead to a reduction in the neuronal damage induced by NMDA even when it was administered alone—i.e. without an external administration of t-PA.

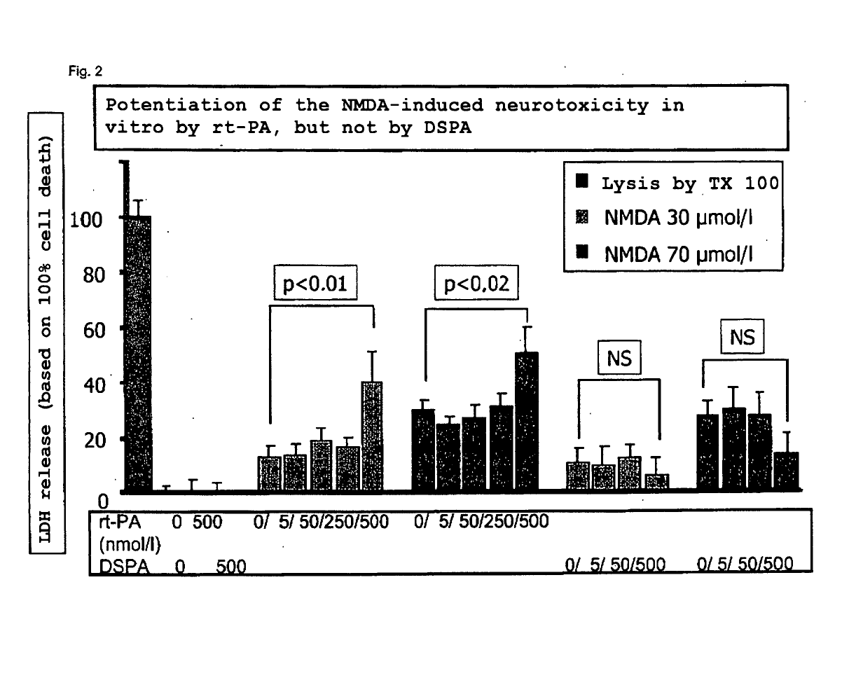

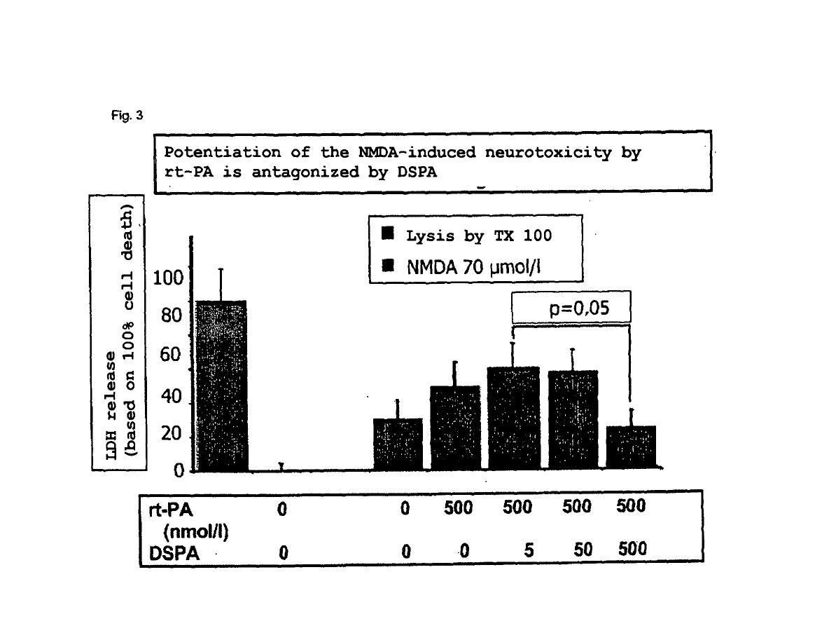

To investigate the neuroprotective effect of DSPA experimentally, cultures of isolated cortical neurons from mouse fetuses were treated with NMDA in order thus to induce neuronal cell death. In addition to NMDA, the culture media contained various amounts of t-PA and/or DSPA. These in vitro experiments show that t-PA brings about a potentiation of the cell death induced by NMDA, whereas DSPA does not cause this effect (FIG. 2). On the other hand, if DSPA was administered together with t-PA, there was a significant reduction in NMDA-induced cell death by comparison with t-PA alone (FIG. 3). It was moreover clear from this that DSPA is capable of a marked concentration-dependent reduction in the neural cell damage which is enhanced by t-PA.

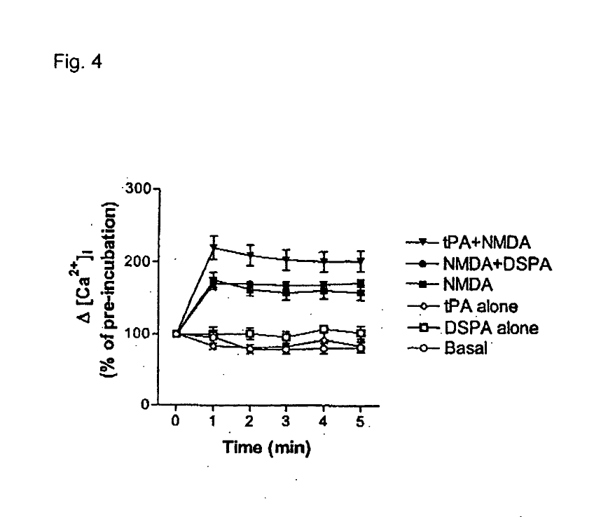

Investigations were further carried out to determine the Ca⁺⁺ influx on previous administration of DSPA or t-PA, because the reduction in the Ca⁺⁺ influx caused by activation of the glutamate receptor is known to be an indicator of the neuroprotection by the respective test substance. The neuroprotection by DSPA was also confirmed in these experiments, because DSPA did not increase the Ca⁺⁺ influx into the cell, and counteracted the damaging effect of the t-PA (FIG. 4).

The mechanism of the neuroprotective effect of DSPA is not yet definitively understood. However, it might derive from the fact that although DSPA, like t-PA, binds to the glutamate receptor, it does not cleave, and thus does not activate, the latter. DSPA would thus not itself contribute to the excitotoxicity and possibly block the activation of the receptor by t-PA.

The suitability of DSPA as neuroprotective is surprising especially in the light of the neurotoxicity of t-PA which has been known since 1995. Since DSPA and t-PA have a considerable functional and structural agreement, it was therefore to be expected that DSPA is also neurotoxic.

A particular advantage of the use according to the invention of DSPA or its derivatives or fragments is based on the fact that, because of the surprisingly found neuroprotective effect of DSPA, it is possible to employ a therapeutic agent that has both fibrinolytic and neuroprotective properties.

This advantage is particularly operative in the treatment of stroke, because the tissue damage associated with stroke is attributable inter alia to the neurotoxic side effects of the endogenous t-PA and of the t-PA which is administered where appropriate for therapeutic purposes. It is possible through the neuroprotective effect of DSPA at least to counteract these damaging effects of t-PA.

In a particularly advantageous embodiment of the invention, DSPA can therefore be administered as neuroprotective in the treatment of stroke in combination with a thrombolytic, for example t-PA. It is thus possible to utilize the therapeutic advantage of t-PA for the patient and at the same time to neutralize, or else at least weaken, its neurotoxic side effects by DSPA as neuroprotective.

The substances which can be employed according to the invention as neuroprotectives can be used for the treatment of a large number of pathological conditions (see above). The possible uses include the treatment of neurodegenerative disorders such as Parkinsonism, Alzheimer's, Huntington's chorea and diabetes, the treatment of painful conditions, the treatment of addictive disorders, the treatment of neurological and psychiatric disorders such as epilepsy, movement disorders, depressions, anxiety states and memory disturbances, a general improvement of cognitive performance and the treatment of amyotrophic lateral sclerosis. Overactivation of the NMDA-glutamate receptor plays a significant part in the pathogenesis of each of these disorders or conditions.

It has already been possible to show the therapeutic efficacy of the neuroprotectives which can be employed according to the invention on the affective state of patients in initial clinical investigations. In this study, the affective state (depressions and anxiety states) of the patients treated with DSPA was examined.

The term “depression” is defined herein comprehensively, i.e. it encompasses all affective or mental disorders which cannot be regarded as an appropriate response to external conditions, irrespective of the physiological or psychological background to their development. The term “depression” includes in particular also anxiety states. Consequently, the term “antidepressant” refers comprehensively to a therapeutic agent for the treatment of these disorders.

The clinical study was carried out to evaluate the clinical effect and safety of DSPA alpha1 in acute ischemic stroke (desmoteplase in acute ischemic stroke, DIAS study hereinafter). This is a placebo-controlled, randomized phase II double-blind study in which DSPA was administered intravenously to the patients within a 3- to 9-hour period after the onset of symptoms of stroke.

46 patients who were selected according to whether their areas of the brain affected by the stroke were irreversibly damaged or else the partially damaged or endangered areas of the brain could still be saved took part in the first part of the study. Only patients in whom previously damaged areas of the brain can potentially be saved by reperfusion with a thrombolytic were included in the study. The patients were selected by MRI (magnetic resonance imaging).

16 patients out of the 46 studied patients were treated with placebo. The other 30 received a desmoteplase product, with 17 patients being treated with 25 mg of the active ingredient and 13 patients with 37.5 or 50 mg.

More than 56% of the placebo-treated patients complained of depression. Although these depressions were clearly diagnosed by the respective treating physician as depression, it was not possible to under-take an unambiguous assignment to a specific manifestation of depression because of the lack of further information. This depressive syndrome was therefore categorized by the treating physicians in each case as “Depression NOS”, namely as “not otherwise specified”.

Accordingly, whereas more than half of the placebo-treated patients showed these unspecified depressions, the proportion of patients affected thereby in the comparable desmoteplase-treated group was overall only 16.7%.

56 patients who were treated inter alia with 62 microg/kg, 90 microg/kg, 125 microg/kg DPSA or with placebo took part in the second part of the study. The results of the second part of the study confirmed the results of the first part both in respect of the reperfusion and in respect of the reduction in the frequency of depression.

Based on these results, a preferred dosage for DSPA alpha1 is greater than 60 and less than 160 microg/kg, in particular from 90 to 125 microg/kg, for the treatment of affective disorders. A reduction in the affective disorders is, however, also possible with higher dosages.

The dosage may differ therefrom on use of other DSPA isoforms or else of DSPA derivatives, analogs or fragments. The dosage is in these cases advantageously adapted on the basis of the respective bioequivalence of the substance used.

This low dosage is advantageous in particular for a depression following stroke (post-stroke depression) when a single treatment takes place by i.v. bolus administration. Even 90 microg/kg DSPA may then be sufficient. Far lower dosages may be adequate on (sub)chronic use. This applies in particular to t-PA inhibitors which are employed according to the invention and lead to suppression of endogenous t-PA production. On the other hand, the abovementioned dosages are preferred for inhibitors which cause a competitive inhibition at the NMDA receptor. Advantageous for less acute pathological states are s.c., oral or inhalational formulations.

The significance of t-PA for the affective state and the learning behavior of humans is known. Thus, t-PA plays a positive part in the learning behavior of healthy people (Pawlak 2002 and 2003). It is likewise known from investigations on t-PA-deficient mouse mutants that t-PA in the amygdala represents a critical factor for stress-induced anxiety states (Pawlak et al. 2003) and is essential in the induction of anxiety (Pawlak 2003). On the other hand, development of depression appears to be connected with the so-called neuronal plasticity with which the body can respond to stress such as, for example, injuries. This process is, according to findings to date, regulated inter alia by the t-PA-mediated glutamate receptor activation.

Viewed together, the known investigations reveal that the endogenous t-PA released through stress or injuries represents a signal for the post-synaptic cell and acts as trigger for plastic neuronal changes.

The mechanism underlying neuroplasticity presumably comprises the so-called long term potentiation (LTP) and the long term depression (LTD). LTP is induced by a high-frequency excitation of the neurons and the increased depolarization induced thereby. This long term potentiation is based on the glutamate-dependent activation of NMDA-glutamate receptors, which leads to an increased Ca⁺⁺ release. The increased calcium concentration brings about an increased efficacy of the synapse and thus permits adaptation to the particular circumstances.

LTD can be induced as “compensation” of a preceding LTP or else de novo by low-frequency electrostimulation. Thus, like LTP, the mechanism is based on activation of glutamate receptors of the NMDA type. However, this leads to a moderate Ca⁺⁺ influx and thus to an only reduced synaptic efficacy. This causes depression.

It is now known that t-PA binds to the glutamate receptor of the NMDA type, and cleaves and thus activates it. This might form the basis for the influence, observed by Pawlak et al., on neuronal plasticity by t-PA. Both the development of a long term potentiation and of a long term depression would be explicable as a function of the amount of t-PA released.

It can thus be inferred from the described investigations on the neuroprotective effect of DSPA according to the invention that DSPA blocks, as antagonist, the activation of the NMDA-glutamate receptor and thus can as a result counteract long term depression.

The difference in the effect of DSPA and t-PA on synaptic activity—and thus on LTP and/or LTD—is also clear from calcium mobilization experiments on neuronal cells (see above). These in vitro experiments show that treatment of neuronal cells with t-PA leads to an NMDA-dependent calcium release, whereas the same treatment with DSPA had no effect on the intracellular calcium concentration (see FIG. 4). The increase in concentration caused by t-PA was 30% compared with the control group.

### EXEMPLARY EMBODIMENTS

I. Clinical Study (DIAS)

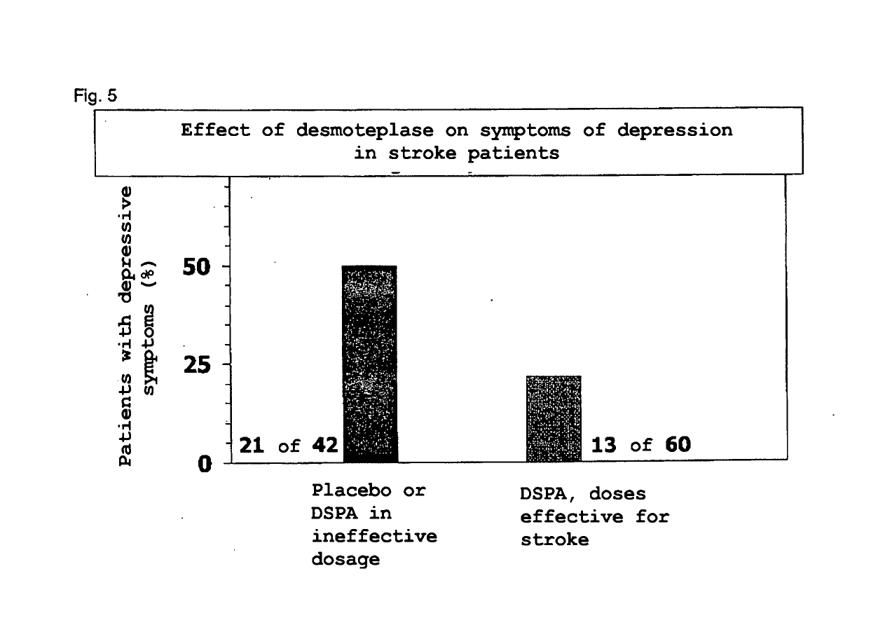

The results of the DIAS study on the effects of DSPA on affective disorders (depression and anxiety states) are summarized in FIG. 5.

II. In Vitro Investigations on Murine Cortical Neurons

Culture of Primary Cortical Mammalian Neurons:

Pregnant Swiss white mice (central animal house of Monash University) were sacrificed on day 14-16 of gestation, and the uteri were removed. The fetuses were removed under sterile conditions, the head was detached, the brain was exposed and the cerebral necortices were dissected out by microdissection under a dissecting microscope (Industrial and Scientific Supply Co.). The dissection was carried out on ice in Hank's balanced salt solution (HBSS; 137 mM NaCl, 5.37 mM KCl, 4.10 mM NaHCO₃, 44 mM KH₂PO₄, 0.13 mM NaHPO₄, 10 mM HEPES, 1 mM pyruvate, 13 mM D(+)glucose and 0.001 g/L phenol red) comprising 3 mg/ml bovine serum albumin (BSA) and 1.2 mM MgSO₄ (pH 7.4). Meninges and blood vessels were carefully removed. The tissue was broken down into small pieces using the tip of a plastic pipette and briefly centrifuged at a rate of 1000 g in order to collect the fragments. The pellet of the fragments was resuspended in warm (37° C.) HBSS (with 3 mg/ml BSA and 1.2 mM MgSO₄) comprising trypsin (0.2 mg/ml) and deoxyribonuclease I (DNase I, 880 U/ml) and incubated in a shaking water bath at 37° C. for 5 min. Digestion of the fragments was stopped by adding an equal volume of HBSS (with 3 mg/ml BSA) comprising trypsin inhibitor (83.2 microg/ml), DNase I (880 U/ml) and MgSO₄ (1.22 mM) and centrifuged at 1000 g for 5 min. The supernatant was aspirated off, and HBSS comprising trypsin inhibitor (0.52 mg/ml), DNase I (880 U/ml) and MgSO₄ (2.7 mM) was added to the pellet. The tissue was dissociated by trituration (15 passes with a caliber 24 needle) and centrifuged at 1000 g for 5 min. The supernatant was aspirated off, and the cells were resuspended in Neurobasal™ medium (InVitrogen, USA; NBM) comprising 2% B27 supplement (Invitrogen, USA), 100 U/ml penicillin and 100 microg/ml streptomycin, 0.5 mM L-glutamine and 10% dialyzed fetal calf serum (dFCS), referred to as complete NBM hereinafter. The cell density of the suspension and culturing yield was found by repeated cell counting in hemocytometer chambers. The cells were in each case inoculated in Nunc™ (Denmark) 24- or 96-sample plates at densities of 0.3×10⁶ or 0.12×10⁶ cells/sample chamber, which was defined as an in vitro time of 0 days (0 div). The plates had previously been coated with poly-D-lysine (50 microg/ml) in order to promote cell adhesion. This coating was removed after overnight incubation at 37° C.

After 24 h (1 cell division), the complete NBM was replaced by dFCS-free complete NBM (2.5% B27 supplement). Half of the serum-free complete NBM was replaced every 3-4 divisions. The cells were kept at 37° C. in a CO₂ humidity incubator and examined by inverted phase contrast microscopy (Olympus, IMT-2). In order to record the morphology of the cell cultures as the cell damage proceeded, photographs were taken (Kodacolor Gold 100 Iso film). All operational steps were carried out at room temperature—unless noted otherwise.

1. Effect of t-PA and DSPA on NMDA-Induced Neuronal Cell Death

An increase in NMDA-induced cell death by t-PA and DSPA was examined by adding NMDA (30 microM or 70 microM) to the cortical neurons cultivated according to the above protocol and the cultures were incubated for a further 24 h. The extent of the induced cell death was measured by determining the liberated lactate dehydrogenase (LDH), using a complete lysis with TX-100 as comparison (FIG. 2). Subsequently, the NMDA was added to the cultures in the presence of in each case increasing concentrations of t-PA (5, 50, 250, 500 nM) or DSPA (5, 50, 500 nM), and the cell death was determined in an analogous manner.

Addition of 30 microM or 70 microM NMDA leads to significant cell death (see FIG. 2) with both NMDA concentrations. In the presence of increasing concentrations of t-PA there is seen to be an increased rate of induced cell death, but only at the highest t-PA concentration of 500 nM (see FIG. 2) in each case. By contrast, no enhancement of the NMDA-induced cell death is to be seen with DSPA, in particular there is even a reduction in the NMDA-induced cell death to be seen at high DSPA concentration. In the absence of NMDA, no neuronal cell death is induced either by t-PA or by DSPA (see FIG. 2).

2. Reduction in the t-PA-Induced Cell Death by DSPA

Further in vitro experiments were carried out to find whether addition of DSPA can counteract the t-PA-induced enhancement of the NMDA-induced cell death. For this purpose, in each case 70 microM NMDA were added to primary cultures of cortical neurons in the presence of a constant t-PA concentration of 500 nM and increasing DSPA concentrations of 5, 50 and 500 nM (FIG. 3). The extent of the cell damage occurring in the cultures is again determined on the basis of the amount of lactate dehydrogenase (LDH) liberated after incubation for 24 h.

In each case NMDA (70 microM) was added to a primary culture of cortical neurons at a constant t-PA concentration of 500 nM and increasing DSPA concentrations (5, 50, 500 nM) and the neuronal cell death was determined on the basis of the LDH amount liberated after 24 h. This revealed an inhibition of the t-PA-dependent stimulation of induced neural cell death with increasing DSPA concentration.

3. Ca⁺⁺ Mobilization Experiment

The intracellular calcium concentration was investigated by an essay using Fluo-3/AM. For this purpose, cortical neurons were cultivated for 9 days (see above) and loaded with 10 microM Fluo-3/AM in HEPES-buffered saline solution. This saline solution comprised 135 mM NaCl, 5 mM KCl, 0.62 mMgSO₄, 1.8 mM CaCl₂, 10 mM HEPES and 6 mM glucose, pH 7.4 at 37° C. for one hour. DMSO (1%) and 0.2% Pluronic F-127 were added in order to facilitate dispersion of the color.

The cells were washed with the abovementioned HEPES buffer, which contained 1 mM furosemide, in order to prevent the color escaping from the buffer (which was employed in every buffer thereafter). The relative fluorescence units (RFU) of the cells were measured after the washing in order thus to obtain a baseline, and during the treatment time (5 minutes), at 485/530 nm (excitation/emission). A fluoroscan ascent fluorometer (Labsystems) was used for this.

As shown in FIG. 4, the NMDA treatment alone caused a significant increase in the calcium concentration after one minute (P<0.05). These calcium concentrations were increased further by 30% when the cells were pretreated with t-PA (30 microg/ml; 500 nM; P<0.05) for 5 minutes before the NMDA was added.

These results are consistent with the report by Nicole et al. 2001, in which previous addition of t-PA further increased the calcium release by NMDA. Treatment with t-PA alone caused no change in the concentration of free calcium ions. Pretreatment of the neuronal cells with DSPA for five minutes before addition of NMDA on the other hand did not change the calcium concentration beyond the extent achieved with addition of NMDA alone. DSPA alone therefore had no effect on the intracellular calcium concentration.

### LIST OF REFERENCES

Chatterton, J. E. et al.: Excitatory glycine receptors containing the NR3 family of NMDA receptor subunits. Nature 415 (2002), 793-798
Das, S. et al.: Increased NMDA current and spine density in mice lacking the NMDA receptor subunit NR3A. Nature 393 (1998), 377-381;
Dennis W. Choi “Glutamat neurotoxicity and diseases of the nervous system”
Gill R. et al.: Pharmacological characterization of RO63-1908 (1-[2-(4-hydroxy-phenoxy)-ethyl]-4-(4-methyl-benzyl)-piperidin-4-01), a novel sinotype-selective N-methyl-D-asparatate antagonist. J. Pharmacol. Exp. Ther. 302 (2002), 940-948
Gotti, B. et al. Ifenprodil and SL 82.0715 as cerebral anti-ischemic agents. Evidence for efficacy in models of focal cerebral ischemia. J. Pharmacoil. Exp. Ther. 247 (1988), 1211-1221
Kemp, J. A. and McKernan R. M.: NMDA Receptor Pathways as Drug Targets, Nature Neuroscience. November 2002, Vol. 5 Suppl., 1039-1042
Kemp, J. A.; Kew, J. N. C.; Gill, R.: Handbook of Experimental Pharmakology Vol. 141 (eds. Jonas, P. & Monyer, H.) 495-527 (Springer, Berlin, 1999);
Lee, Jin-Moo et al.: “The changing landscape of ischemic brain injury mechanisms”;
Lees, K. R. et al.: Glycine antagonist (gavestinel) in neuroprotection (GAIN International) in patients with acute STROKE: a randomised controlled trial. GAIN International Investigators. Lancet 355 (2000), 1949-1954;
Nicole O., Docagne F., Ali C., Margaill I., Carmeliet P., MacKenzie E. T., Vivien D., Buisson A., 2001: The proteolytic activity of tissue-plasminogen activator enhances NMDA receptor-mediated signaling, Nat. Med. 7, 59-64
Pawlak R., Magarinos A. M., Melchor J., McEwen B., Strickland S., 2003: Tissue plasminogen activator in the amygdala is critical for stress-induced anxiety-like behavior, nature neuroscience, 6 (2), 168-174
Pawlak R., Nagal N., Urano T., Napiorkowska-Pawlak D., Ihara H., Takada Y., Collen D. Takada A., Rapid, specific and active site-catalyzed effect of tissue-Plasminogen activator on hippocampus-dependent learning in mice, 2002, Neuroscience, 113(4), 995-1001
Press release from Pfizer, October 2001).
Sacco, R. L. et al. Glycine antagonist in neuroprotection for patients with acute stroke: GAIN Americas: a randomized controlled trial. JAMA 285 (2001), 1719-1728

1-11. (canceled)

12. A method for the treatment of depression or an anxiety state in a human in need thereof, comprising administering to said human an effective amount of an inhibitor of the t-PA-mediated activation of a glutamate receptor.

13. A method according to claim 12, wherein the glutamate receptor is of the NMDA type.

14. A method according to claim 12, wherein the inhibitor is a protease.

15. A method according to claim 14, wherein the protease is a serine protease inhibitor.

16. A method according to claim 15, wherein the serine protease inhibitor is chosen from neuroserpin, plasminogen activator inhibitor (PAI), and protease nexin I (PN-1).

17. A method according to claim 12, wherein the inhibitor is chosen from DSPA and a DSPA derivative, analog, or fragment.

18. A method according to claim 17, wherein the sequence of the DSPA or DSPA derivative, analog, or fragment is the amino acid sequence of SEQ ID NO:1 or has at least 70% homology with the sequence of SEQ ID NO:1.

19. A method according to claim 18, wherein the sequence of the DSPA or DSPA derivative, analog, or fragment has from 80 to 90% homology with the sequence of SEQ ID NO:1.

20. A method according to claim 17,
wherein a DSPA is administered to a human in a dose greater than 60 and lower than 160 microgram/kg;
wherein the DSPA has the amino acid sequence of SEQ ID NO:1.

21. A method according to claim 17,
wherein a DSPA or a DSPA derivative, analog, or fragment is administered to a human in a dose from 90 to 125 microgram/kg;
wherein the DSPA has the amino acid sequence of SEQ ID NO:1.

22. A method according to claim 17,
wherein a DSPA or a DSPA derivative, analog, or fragment is administered to a human in a dose of about 90 microgram/kg;
wherein the DSPA has the amino acid sequence of SEQ ID NO:1.

23. A method for the treatment of stroke in a human in need thereof comprising administering to said human an effective amount of a DSPA or a DSPA derivative, analog, or fragment and a thrombolytic.

24. A method according to claim 23, wherein the thrombolytic is t-PA.

25. A method for providing neuroprotection in a human in need thereof comprising administering to said human an effective amount of a DSPA or a DSPA derivative, analog, or fragment.

26. A method according to claim 25, wherein the method for providing neuroprotection is a method for the treatment or prophylaxis of a condition chosen from Parkinsonism, Alzheimer's, Huntington's chorea, diabetes, painful conditions, epilepsy, and memory disturbances.

27. A method according to claim 17, wherein the DSPA derivative, analog, or fragment is administered to a human in a dose bioequivalent to the administration of a dose greater than 60 and lower than 160 microgram/kg of a DSPA having the amino acid sequence of SEQ ID NO:1.

28. A method according to claim 17, wherein the DSPA derivative, analog, or fragment is administered to a human in a dose bioequivalent to the administration of a dose from 90 to 125 microgram/kg of a DSPA having the amino acid sequence of SEQ ID NO:1.

29. A method according to claim 17, wherein the DSPA derivative, analog, or fragment is administered to a human in a dose bioequivalent to the administration of a dose of about 90 microgram/kg of a DSPA having the amino acid sequence of SEQ ID NO:1.


SEQUENCE LISTING (1 sequence)

SEQ ID NO: 1 - protein, 477 aa, Desmodus rotundus
MVNTMKTKLLCVLLLCGAVFSLPRQETYRQLARGSRAYGVACKDEITQMTYRRQESWLRPEVRSKRVEHCQCDRGQARCHTVPVNSCSEPRCFNGGTCWQAVYFSDFVCQCPAGYTGKRCEVDTRATCYEGQGVTYRGTWSTAESRVECINWNSSLLTRRTYNGRMPDAFNLGLGNHNYCRNPNGAPKPWCYVIKAGKFTSESCSVPVCSKATCGLRKYKEPQLHSTGGLFTDITSHPWQAAIFAQNRRSSGERFLCGGILISSCWVLTAAHCFQESYLPDQLKVVLGRTYRVKPGEEEQTFKVKKYIVHKEFDDDTYNNDIALLQLKSDSPQCAQESDSVRAICLPEANLQLPDWTECELSGYGKHKSSSPFYSEQLKEGHVRLYPSSRCAPKFLFNKTVTNNMLCAGDTRSGEIYPNVHDACQGDSGGPLVCMNDNHMTLLGIISWGVGCGEKDVPGVYTKVTNYLGWIRDNMHL